(19) United States
(12) Patent Application Publication
(10) Pub. No.: US 2025/0041517 A1
(43) Pub. Date: Feb. 6, 2025

(54) Pressure Threshold Detector for Medical Injector

(71) Applicant: Becton, Dickinson and Company, Franklin Lakes, NJ (US)

(72) Inventor: David James Coleman, Shankill (IE)

(21) Appl. No.: 18/718,872

(22) PCT Filed: Dec. 13, 2022
(86) PCT No.: PCT/US22/52655

§ 371 Date: Jun. 12, 2024

Related U.S. Application Data
(60) Provisional application No. 63/288,967, filed on Dec. 13, 2021.

(51) Int. Cl. A61M 5/168

(52) U.S. Cl. CPC A61M 5/16859; A61M 2205/106; A61M 2205/50

(57) ABSTRACT

A pressure threshold detector for a medical injector includes a first member, a second member moveable relative to the first member between a retracted position and an extended position, an inlet passageway configured to be in fluid communication with a fluid line, a cantilever arm having a cam surface, and a protrusion having a first cam profile and a second cam profile. The cam surface of the cantilever arm is engaged with the first cam profile of the protrusion when the second member is in the retracted position, and the cam surface of the cantilever arm is engaged with the second cam profile of the protrusion when the second member is in the extended position. The second member is moveable from the retracted position to the extended position when a pressure within the inlet passageway exceeds a predetermined pressure.

### CROSS-REFERENCE TO RELATED APPLICATION

The present application claims priority to U.S. Provisional Application Ser. No. 63/288,967, entitled “Pressure Threshold Detector for Medical Injector” filed Dec. 13, 2021, the entire disclosure of which is hereby incorporated by reference in its' entirety.

### BACKGROUND OF THE INVENTION

### Field of the Invention

The present disclosure relates to a pressure threshold detector for a medical injector

### Description of Related Art

Wearable medical devices, such as automatic injectors, have the benefit of providing therapy to the patient at a location remote from a clinical facility and/or while being worn discretely under the patient's clothing. The wearable medical device can be applied to the patient's skin and configured to automatically deliver a dose of a pharmaceutical composition within a predetermined time period after applying the wearable medical device to the patient's skin, such as after a 27 hour delay. After the device delivers the pharmaceutical composition to the patient, the patient may subsequently remove and dispose of the device.

In certain circumstances, due to the medium in which the liquid is being injected, the flow of fluid leaving the device may be impaired, which can lead to increased pressure in the fluid line of the device. When the pressure rises above a certain threshold, the integrity of the fluid path may be compromised causing a leak within the device and a failure to deliver the full dose of medicament. A fluid leak within the device may also cause damage to the device and subsequent system failures as well as potential contamination concerns due to contact between the fluid and the device.

Human subcutaneous tissue is composed of various cell types, extracellular matrix (ECM) constituents, microstructures, and macroscopic arrangement of cells and ECM. Those elements contribute to the mechanical properties of the tissue. The tissue may also include lymphatic system and blood vessels, and have intrinsic fluid absorption and retention properties. These characteristics vary among individuals, location within the body, and over time may cause variable degrees of resistance to the infusion of fluids at the site of injection. When the resistance of the tissue is too high or the absorption rate is too low for a given delivery flow rate from the device, the pressure may build up and reach valves above the threshold where the fluid line and other components may be compromised.

### SUMMARY OF THE INVENTION

In one aspect or embodiment, a pressure threshold detector for a medical injector includes a first member having a first end and a second end positioned opposite the first end, a second member having a first end and a second end positioned opposite the first end, with the second member moveable relative to the first member between a retracted position and an extended position, an inlet passageway configured to be in fluid communication with a fluid line, a cantilever arm having a cam surface, and a protrusion having a first cam profile and a second cam profile. The cam surface of the cantilever arm is engaged with the first cam profile of the protrusion when the second member is in the retracted position, and the cam surface of the cantilever arm is engaged with the second cam profile of the protrusion when the second member is in the extended position. The cantilever arm is positioned on one of the first member and the second member and the protrusion is positioned on the other of the first member and the second member. The second member is moveable from the retracted position to the extended position when a pressure within the inlet passageway exceeds a predetermined pressure.

The pressure threshold detector may include a stop surface positioned adjacent to the second cam profile, with the stop surface configured to engage the cam surface of the cantilever arm to restrict movement of the second member relative away from the first member. The pressure threshold detector may include a switch, with the second member engaged with the switch when the second member is in the extended position. The first member may define an interior, with at least a portion of the second member received within the interior. The pressure threshold detector may include a seal positioned within the interior between the first member and the second member, with the first member defining the inlet passageway and the first member and the second member defining a pressure space between the seal and the inlet passageway.

The cantilever arm may be positioned on the first member and the protrusion may be positioned on the second member, with the cam surface of the cantilever arm extending radially inward and the first and second cam profiles extending radially outward. The cam surface may be semi-spherical and the first and second cam profiles may each be frustoconical.

The cantilever arm may be configured to be biased radially outward as the second member moves from the retraced position to the extended position, with the cantilever arm biasing the second member toward the extended position when the cam surface of the cantilever arm is engaged with the second cam profile of the protrusion. The cantilever arm may be formed integrally with the first member and the protrusion may be formed integrally with the second member. The cantilever arm may extend from the first end of the first member. The protrusion may be positioned between the first and second ends of the second member. The predetermined pressure may be 5-25 psi.

In a further aspect or embodiment, a medical injector includes a power source, a reservoir configured to receive a fluid, a fluid line in fluid communication with the reservoir, a pump configured to deliver a fluid from the reservoir to the fluid line, and a pressure threshold detector of any of the aspects or embodiments discussed above, with the inlet passageway of the pressure threshold detector in fluid communication with the fluid line.

The medical injector may further include a microcontroller, with the pressure threshold detector sending a signal to the microcontroller when the second member is in the extended position.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a medical injector according to one aspect or embodiment of the present application;

FIG. 2 is a perspective view of the medical injector of FIG. 1, with a top cover removed;

FIG. 3 is a schematic of the medical injector of FIG. 1;

FIG. 4 is a front view of a pressure threshold detector according to one aspect or embodiment of the present application; and

FIG. 5 is a cross-sectional view of the pressure threshold detector of FIG. 4.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

All numbers used in the specification and the claims are to be understood as being modified in all instances by the term “about”. By “about” is meant a range of plus or minus ten percent of the stated value. As used in the specification and the claims, the singular form of “a”, “an”, and “the” include plural referents unless the context clearly dictates otherwise. The terms “first”, “second”, and the like are not intended to refer to any particular order or chronology, but instead refer to different conditions, properties, or elements. By “at least” is meant “greater than or equal to”.

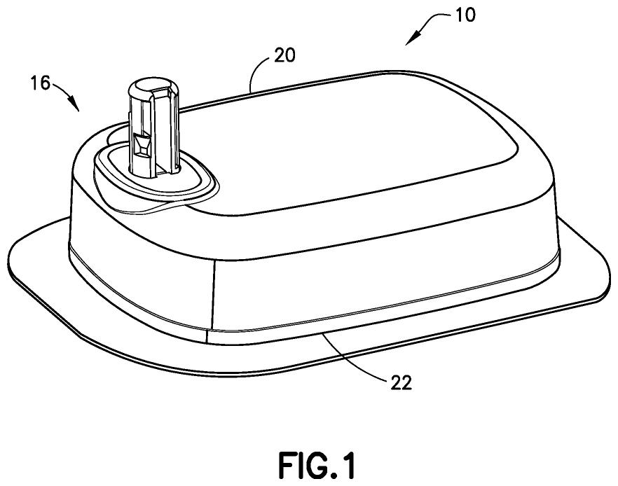

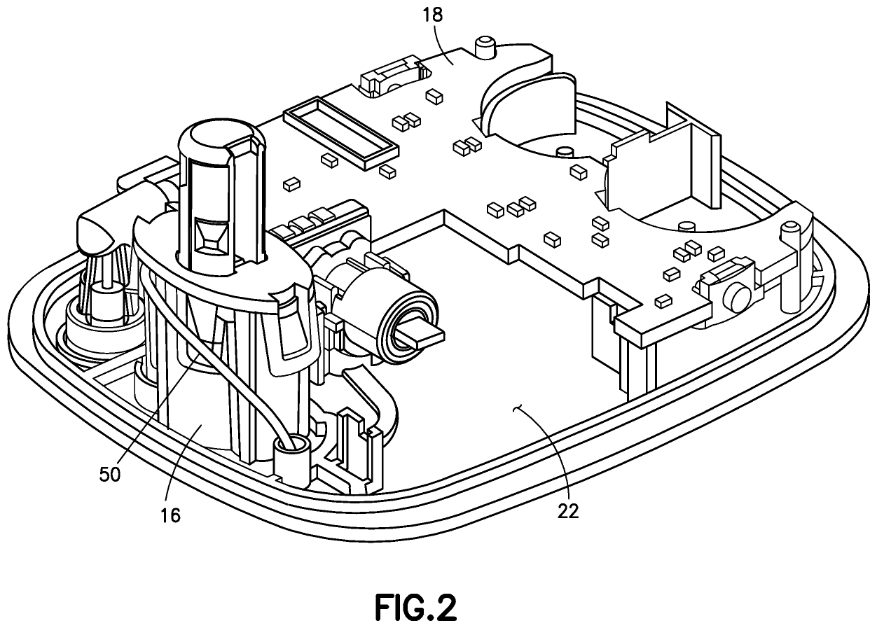

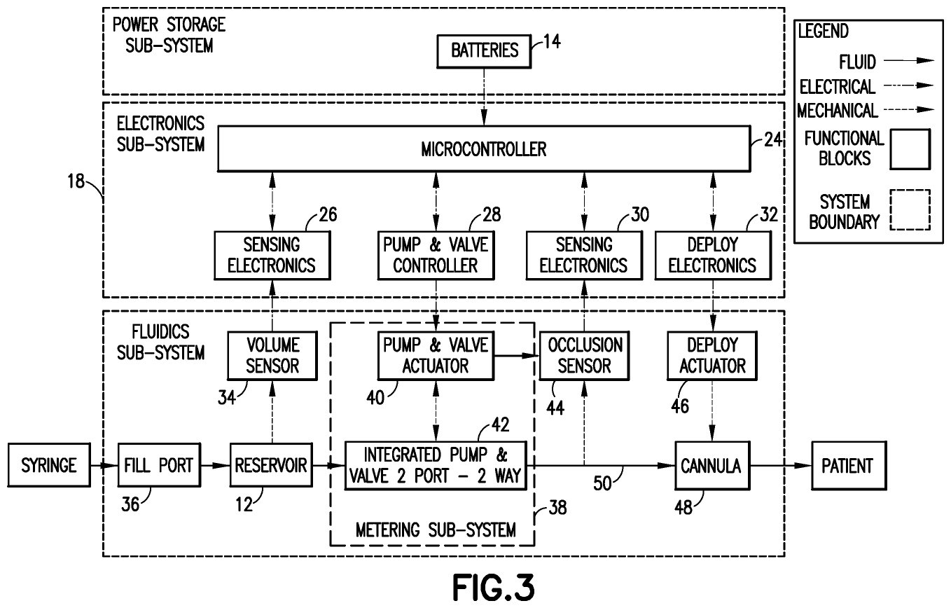

Referring to FIGS. 1-5, a medical injector 10 includes a reservoir 12, a power source 14, an insertion mechanism 16, control electronics 18, a cover 20, and a base 22. In one aspect or embodiment, the medical injector 10 is a wearable automatic injector, such as an insulin or bone marrow stimulant delivery device. The medical injector 10 may be mounted onto the skin of a patient and triggered to inject a pharmaceutical composition from the reservoir 12 into the patient. The medical injector 10 may be pre-filled with the pharmaceutical composition, or it may be filled with the pharmaceutical composition by the patient or medical professional prior to use.

The medical injector 10 is configured to deliver a dose of a pharmaceutical composition, e.g., any desired medicament, into the patient's body by a subcutaneous injection at a slow, controlled injection rate. Exemplary time durations for the delivery achieved by the medical injector 10 may range from about 5 minutes to about 60 minutes, but are not limited to this exemplary range. Exemplary volumes of the pharmaceutical composition delivered by the medical injector 10 may range from about 0.1 milliliters to about 10 milliliters, but are not limited to this exemplary range. The volume of the pharmaceutical composition delivered to the patient may be adjusted.

Referring again to FIGS. 1-5, in one aspect or embodiment, the power source 14 is a DC power source including one or more batteries. The control electronics 18 include a microcontroller 24, sensing electronics 26, a pump and valve controller 28, sensing electronics 30, and deploy electronics 32, which control the actuation of the medical injector 10. The medical injector 10 includes a fluidics sub-system that includes the reservoir 12, a volume sensor 34 for the reservoir 12, a reservoir fill port 36, and a metering system 38 including a pump and valve actuator 40 and a pump and valve mechanism 42. The fluidic sub-system may further include an occlusion sensor 44, a deploy actuator 46, a cannula 48 for insertion into a patient's skin, and a fluid line 50 in fluid communication with the reservoir 12 and the cannula 48. In one aspect or embodiment, the insertion mechanism 16 is configured to move the cannula 48 from a retracted position positioned entirely within the device 10 to an extended position where the cannula 48 extends outside of the device 10. The medical injector 10 may operate in the same manner as discussed in U.S. Pat. No. 10,449,292 to Pizzochero et al. As discussed in more detail below, in one aspect or embodiment, the medical injector 10 includes a pressure threshold detector 52 in fluid communication with the reservoir 12, the pump and valve actuator 40, and/or the fluid line 50. In one aspect or embodiment, the pressure threshold detector 52 forms part of the occlusion sensor 44.

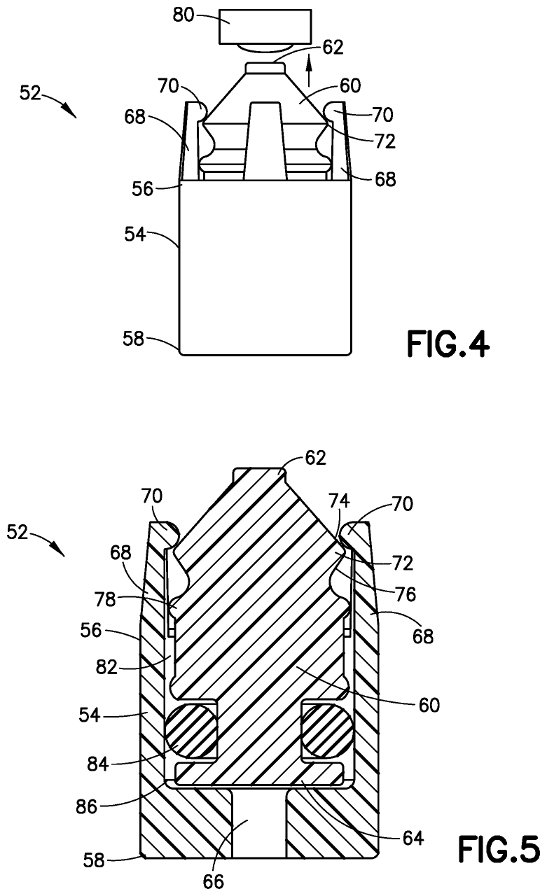

Referring to FIGS. 4 and 5, the pressure threshold detector 52 includes a first member 54 having a first end 56 and a second end 58 positioned opposite the first end 56, a second member 60 having a first end 62 and a second end 64 positioned opposite the first end 62, with the second member 60 moveable relative to the first member 54 between a retracted position and an extended position, an inlet passageway 66 configured to be in fluid communication with the fluid line 50, a cantilever arm 68 having a cam surface 70, and a protrusion 72 having a first cam profile 74 and a second cam profile 76. The cam surface 70 of the cantilever arm 68 is engaged with the first cam profile 74 of the protrusion 72 when the second member 60 is in the retracted position. The cam surface 70 of the cantilever arm 68 is engaged with the second cam profile 76 of the protrusion 72 when the second member 60 is in the extended position. As shown in FIGS. 4 and 5, the cantilever arm 68 is positioned on the first member 54 and the protrusion 72 is positioned on the second member, although the cantilever arm 68 may be positioned on the second member 60 and the protrusion 72 positioned on the first member 54. The second member 60 is moveable from the retracted position to the extended position when a pressure within the inlet passageway 66 exceeds a predetermined pressure. The cam surface 70 of the cantilever arm 68 extends radially inward and the first and second cam profiles 74, 76 extend radially outward.

In one aspect or embodiment, the cantilever arm 68 includes one or more cantilever arms 68. In one aspect or embodiment, the cantilever arm 68 includes four equally-spaced cantilever arms 68. In one aspect or embodiment, the protrusion extends circumferentially around the second member 60.

Referring again to FIGS. 4 and 5, the pressure threshold detector 52 also includes a stop surface 78 positioned adjacent to the second cam profile 76, with the stop surface 78 configured to engage the cam surface 70 of the cantilever arm 68 to restrict movement of the second member 60 relative away from the first member 54. In particular, the stop surface 78 engages the cam surface 70 to prevent pressure within the inlet passageway 66 from forcing the second member 60 entirely away from the first member 54, which would cause the pressure threshold detector 52 to leak. In one aspect or embodiment, the pressure threshold detector 52 includes a switch 80, with the second member 60 engaged with the switch 80 when the second member 60 is in the extended position. The switch may be electrical, mechanical, or electromechanical and may be configured to send a signal to the medical injector 10, such as the microcontroller 24 of the medical injector 10. The first member 54 defines an interior 82, with at least a portion of the second member 60 received within the interior 82. A seal 84 is positioned within the interior 82 between the first member 54 and the second member 60, with the first member 54 defining the inlet passageway 66, and with the first member 54 and the second member 60 defining a pressure space 86 between the seal 84 and the inlet passageway 66. The seal 84 may be an elastomeric O-ring, although other suitable seals may be utilized.

The cam surface 70 is semi-spherical and the first and second cam profiles 74, 76 are each frustoconical, although other suitable shapes and arrangements may be utilized. The cantilever arm 68 is configured to be biased radially outward as the second member 60 moves from the retraced position to the extended position, with the cantilever arm 68 biasing the second member 60 toward the extended position when the cam surface 70 of the cantilever arm 68 is engaged with the second cam profile 76 of the protrusion 72. As the pressure within the pressure space 86 increases, the second member 60 will move in a direction away from the first member 54 until the cam surface 70 passes over the transition between the first cam profile 74 and the second cam profile 76, with the cam surface 70 then engaging the second cam profile 76 to move the second member 60 to the extended position. The distance the second member 60 moves relative to the first member 54 may be the distance between the first cam profile 74 and the stop surface 78. The second member 60 will move to the retracted position when the pressure within the inlet passageway exceeds the reaction force between the cam surface 70 and the first cam profile 74 and the seal 84 joint pressure force.

The cantilever arm 68 is formed integrally with the first member 54 and the protrusion 72 is formed integrally with the second member 60. In one aspect or embodiment, the cantilever arm 68 and the first member 54, and the protrusion 72 and the second member 60, are each plastic-injection molded. The cantilever arm 68 extends from the first end 56 of the first member 54 and the protrusion 72 is positioned between the first and second ends 62, 64 of the second member 60.

In one aspect or embodiment, the predetermined pressure within the inlet passageway 66 where the second member 60 moves from the retracted position to the extended position is 5-25 psi.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A pressure threshold detector for a medical injector comprising:
a first member having a first end and a second end positioned opposite the first end;
a second member having a first end and a second end positioned opposite the first end, the second member moveable relative to the first member between a retracted position and an extended position;
an inlet passageway configured to be in fluid communication with a fluid line;
an arm having a cam surface; and
a protrusion having a first cam profile and a second cam profile,
wherein the cam surface of the arm is engaged with the first cam profile of the protrusion when the second member is in the retracted position, the cam surface of the arm is engaged with the second cam profile of the protrusion when the second member is in the extended position, the arm positioned on one of the first member and the second member and the protrusion positioned on the other of the first member and the second member, and wherein the second member is moveable from the retracted position to the extended position when a pressure within the inlet passageway exceeds a predetermined pressure.

2. The pressure threshold detector of claim 1, wherein the arm is a cantilever arm.

3. The pressure threshold detector of claim 1, wherein the arm applies a force to translate the second member from the retracted position to the extended position.

4. The pressure threshold detector of claim 1, further comprising a stop surface positioned adjacent to the second cam profile, the stop surface configured to engage the cam surface of the arm to restrict movement of the second member relative away from the first member.

5. The pressure threshold detector of claim 1, further comprising a switch, the second member engaged with the switch when the second member is in the extended position.

6. The pressure threshold detector of claim 1, wherein the first member defines an interior, at least a portion of the second member received within the interior.

7. The pressure threshold detector of claim 6, further comprising a seal positioned within the interior between the first member and the second member, wherein the first member defines the inlet passageway, and wherein the first member and the second member define a pressure space between the seal and the inlet passageway.

8. The pressure threshold detector of claim 7, wherein the arm is positioned on the first member and the protrusion is positioned on the second member, the cam surface of the arm extending radially inward and the first and second cam profiles extending radially outward.

9. The pressure threshold detector of claim 1, wherein the cam surface is semi-spherical.

10. The pressure threshold detector of claim 1, wherein the first and second cam profiles are each frustoconical.

11. The pressure threshold detector of claim 1, wherein the arm is configured to be biased radially outward as the second member moves from the retraced position to the extended position, and wherein the arm biases the second member toward the extended position when the cam surface of the arm is engaged with the second cam profile of the protrusion.

12. The pressure threshold detector of claim 1, wherein the arm is formed integrally with the first member and the protrusion is formed integrally with the second member.

13. The pressure threshold detector of claim 12, wherein the arm extends from the first end of the first member.

14. The pressure threshold detector of claim 13, wherein the protrusion is positioned between the first and second ends of the second member.

15. The pressure threshold detector of claim 1, wherein the predetermined pressure is 5-25 psi.

16. A medical injector comprising:
a power source;
a reservoir configured to receive a fluid;
a fluid line in fluid communication with the reservoir;
a pump configured to deliver a fluid from the reservoir to the fluid line; and
a pressure threshold detector of claim 1, the inlet passageway of the pressure threshold detector in fluid communication with the fluid line.

17. The medical injector of claim 16, further comprising a microcontroller, wherein the pressure threshold detector sends a signal to the microcontroller when the second member is in the extended position.
